(19) United States
(12) Patent Application Publication
(10) Pub. No.: US 2020/0147265 A1
(43) Pub. Date: May 14, 2020

(54) METHOD FOR CROSSLINKING ARTIFICIAL BIOLOGICAL TISSUE

(71) Applicant: Peijia Medical (Suzhou) Co., Ltd., Suzhou (CN)

(72) Inventors: Kongrong Karl PAN, Suzhou (CN); Yi Zhang, Suzhou (CN); Guozheng Cheng, Suzhou (CN); Jinjin Zhang, Suzhou (CN); Kun Zhang, Suzhou (CN)

(73) Assignee: Peijia Medical (Suzhou) Co., Ltd.

(21) Appl. No.: 16/414,188

(22) Filed: May 16, 2019

(30) Foreign Application Priority Data
Nov. 8, 2018 (CN) 201811326745.1

(51) Int. Cl. A61L 27/36; A61L 27/60; A61F 2/24; A61F 2/08; A61F 2/10

(52) U.S. Cl. CPC A61L 27/3687; A61L 27/60; A61L 2400/02; A61F 2/08; A61F 2/105; A61F 2/2475

(57) ABSTRACT

The present disclosure provides a method for crosslinking an artificial biological tissue. The method may include: providing an artificial biological tissue and crosslinking agent solution, wherein the crosslinking agent includes an imide structure; immersing the artificial biological tissue into the crosslinking agent solution to produce a crosslinking reaction. In this way, the anti-calcification capacity of the crosslinked artificial biological tissue may be improved in the present disclosure.

### CROSS-REFERENCE TO RELATED APPLICATIONS

This application claims priority to Chinese Patent Application No. 201811326745.1, filed on Nov. 8, 2018, the contents of which is herein incorporated by reference in its entirety.

### TECHNICAL FIELD

The present disclosure relates to a biomedical technology, and more particularly, to a method for crosslinking an artificial biological tissue.

### BACKGROUND

With the development of economy and aging of population, senile calcific aortic valve disease shows a rising trend in morbidity, and has become a cardiovascular disease that ranks after coronary heart disease and hypertension. A retrospective and non-random study and analysis indicates that, among the middle and old age patients, the morbidity of calcific aortic valve disease is as high as 49.38%. With the aging of the population, the morbidity of calcific aortic valve disease increases, and calcific aortic valve disease will become a leading cause of valvular heart disease in China.

In 1965, Carpentier was the first to use glutaraldehyde to treat biological materials, and the biological valve treated showed enhanced durability. Valve or pericardial tissue was treated with glutaraldehyde solution alone to undergo crosslinking. Under certain conditions, the valve or pericardial tissue is provided with mechanical property and durability within a range of the present disclosure, and also provided with fine biocompatibility.

During a long research, it is found that in a living collagen fiber of a natural valve, glycoprotein with mucopolysaccharide masks and prohibits collagens from binding calcium and phosphorus to form a crystal core, therefore calcification is rarely observed. However, when the biological valve has undergone crosslinking with glutaraldehyde, the valve often includes unmatched aldehyde groups from the glutaraldehyde and carboxylic groups from valve protein tissue, which are susceptible to binding calcium ions, thereby generating calcification sites within the biological valve. This is an important cause of biological valve calcification.

### SUMMARY

A technical problem mainly to be solved by the present disclosure is to provide a method for crosslinking an artificial biological tissue to enhance the anti-calcification capacity of the crosslinked artificial biological tissue.

In order to solve the above-mentioned technical problem, a technical solution adopted by the present disclosure is to provide a method for crosslinking an artificial biological tissue, including: providing an artificial biological tissue and crosslinking agent solution, wherein the crosslinking agent solution comprises an imide structure; and immersing the artificial biological tissue into the crosslinking agent solution to produce a crosslinking reaction.

The advantageous effects of the disclosure include, beyond the state of the related art, providing a method for crosslinking an artificial biological tissue, wherein the crosslinking agent used in the crosslinking method is a crosslinking agent with an imide structure, such that no calcification sites are generated, the anti-calcification capacity of the artificial biological tissue is enhanced after the crosslinking reaction, and the artificial biological tissue after the crosslinking reaction shows better physical and chemical property, as well as better biocompatibility.

### BRIEF DESCRIPTION OF THE DRAWINGS

FIG. 1 is a schematic flowchart of a method for crosslinking an artificial biological tissue according to a first embodiment of the present disclosure.

FIG. 2 is a performance parameter comparison chart of a maximum stress of the crosslinked artificial biological tissue according to the present disclosure.

FIG. 3 is a performance parameter comparison chart of a tensile strength of the crosslinked artificial biological tissue according to the present disclosure.

FIG. 4 is a performance parameter comparison chart of an elasticity modulus of the crosslinked artificial biological tissue according to the present disclosure.

FIG. 5 is an anti-calcification performance parameter comparison chart of the crosslinked artificial biological tissue according to the present disclosure.

### DETAILED DESCRIPTION

The detailed description set forth below is intended as a description of the subject technology with reference to the appended figures and embodiments. It is understood that the embodiments described herein include merely some parts of the embodiments of the present disclosure, but do not necessarily include all the embodiments. Based on the embodiments of the present disclosure, all other embodiments that those skilled in the art may derive from these embodiments are within the scope of the present disclosure.

The present disclosure provides a method for crosslinking an artificial biological tissue, and non-glutaraldehyde solution is used in the method, therefore the susceptibility of calcification of the glutaraldehyde crosslinked artificial biological tissue is avoided.

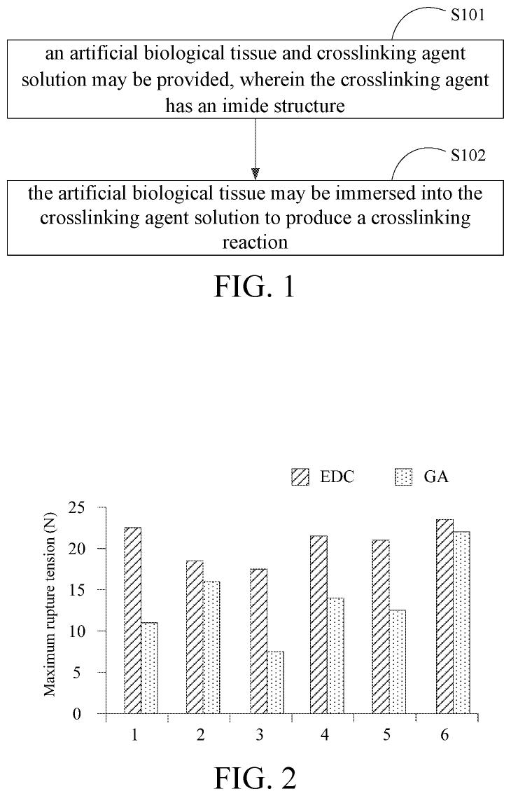

, FIG. 1 is a schematic diagram of the first embodiment of the method for crosslinking an artificial biological tissue according to the present disclosure. In this embodiment, the crosslinking method includes operations in the following blocks.

Block S101, an artificial biological tissue and crosslinking agent solution is provided.

The artificial biological tissue may be a mammal tissue, and the mammal tissue may be any one of an animal pericardium, an aortic valve, a mitral valve, a tricuspid valve, a pulmonary valve, a ligament, a skin, a peritoneum, a pleura, a heel tendon or a venous valve, or a mixture of at least two selected from the above. This method may be applied in artificial biological valves such as aortic, mitral, tricuspid, pulmonary valves, and the like.

Block S102, the artificial biological tissue is immersed in the crosslinking solution to produce a crosslinking reaction.

The crosslinking agent may include an imide structure. In this embodiment, a non-aldehyde crosslinking agent may be employed to crosslink biological tissues, and the crosslinked biological tissue thus obtained may show better physical and chemical property, better biocompatibility and better anti-calcification capability.

In one embodiment, the crosslinking agent solution may be a mixed solution of 1-ethyl-3-(3-dimethylaminopropyl) carbodiimide (EDC) and N-hydroxy succinimide (NHS).

In at least one embodiment, carbodiimide (EDC) may be a chemical crosslinking agent which is often used with N-hydroxy succinimide (NHS) or N-hydroxy thiosuccinimide to enhance the coupling efficiency. The crosslinking agent solution may be obtained by providing a predetermined amount of 1-(3-dimethylaminopropyl)-3-ethylcarbodiimide hydrochloride (EDC.HCl) and N-hydroxy succinimide (NHS), mixing and dissolving them in water.

The mass fraction of 1-ethyl-3-(3-dimethylaminopropyl) carbodiimide (EDC) in the crosslinking agent solution may be 0.5 g/L˜30 g/L, such as 0.5 g/L, 1 g/L, 2 g/L, 3 g/L, 4 g/L, 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L or 30 g/L, or it may be any particular value between any of the above values. The present disclosure does not list all the particular values included in the range.

The mass fraction of N-hydroxy succinimide (NHS) in the crosslinking agent solution may be 0.1 g/L˜10 g/L, such as 0.1 g/L, 1 g/L, 2 g/L, 3 g/L, 4 g/L, 5 g/L, 6 g/L, 7 g/L, 8 g/L, 9 g/L or 10 g/L, or it may be any particular value between any of the above values. The present disclosure does not list all the particular values included in the range.

After the crosslinking agent solution is prepared, the biological tissue may be immersed in the crosslinking agent solution to crosslink. The crosslinking may be carried out under a certain pressure. For example, the biological tissue may be tightened onto a pre-set device and a drawing force is applied onto the tissue. The crosslinking pressure may be 0 mmHg-40 mmHg, such as 0 mmHg, 5 mmHg, 10 mmHg, 15 mmHg, 20 mmHg, 25 mmHg, 30 mmHg, 35 mmHg, 40 mmHg, or it may be any particular value between any of the above values. The present disclosure does not list all the particular values included in the range. By applying a certain pressure, the crosslinking speed and degree may be enhanced.

The crosslinking may be reacted by a crosslinking machine under a certain mechanical motion. The crosslinking mechanical motion may have motion amplitude in a range of 10 rpm to 100 rpm, such as 10 rpm, 20 rpm, 30 rpm, 40 rpm, 50 rpm, 60 rpm, 70 rpm, 80 rpm, 90 rpm, 100 rpm, or any particular value between the above values. The present disclosure does not list all the particular values included in the range. By applying a mechanical movement, the crosslinking between parts of the biological tissue may be more uniform.

The crosslinking may be reacted under a predetermined range of temperature, and the crosslinking temperature may be 25° C.˜45° C., such as 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., or it could be any particular value between any of the above values. The present disclosure does not list all the particular values included in the range. If the temperature is too low, the crosslinking speed would be too slow. If the temperature is too high, the biological tissue would be damaged.

When the crosslinking condition is determined, the crosslinking time may be chosen according to the type of biological tissues and requirements on the crosslinking degree of the products. The crosslinking time may be 0.5-60 days, such as 0.5 day, 1 day, 2 days, 4 days, 8 days, 12 days, 16 days, 20 days, 24 days, 32 days, 40 days, 48 days or 60 days, or it could be any particular value between any of the above values. The present disclosure does not list all the particular values included in the range.

When the crosslinking is over, performance tests and storage will be carried out according to the needs.

In the following descriptions, the solution of the present disclosure shall be explained and illustrated in several particular experimenting examples. The experimental examples are merely some exemplary solutions and should not be used to limit the scope of the present disclosure. Where the experimental examples do not indicate the particular conditions of the experimental methods, conditions indicated by regular conditions or standard requirements would be adopted. Unless otherwise defined, all professional and scientific terms used herein may have the same meanings as the meanings well known by those skilled in the art.

Some reference standards in the industry may be applied in the following experimental examples. Please refer to relevant documents for the context of the standards. The standards may include as following descriptions:

GB/T 528-2009 Rubber, vulcanized rubber or thermoplastic, determination of tensile stress-strain properties.

EP130015 Measuring experiment protocol of cardiac vesicle crosslinking degree (X1 version).

YY/T 1449.3-2016 Cardiovascular implant—Artificial heart valve Part III, Transcatheter implantable prosthetic heart valve.

The detecting methods in Section 5.2.2, GB/T 14233.1-2008.

The detecting methods in Section 8.2, GB/T16886.5-2003.

TP 13010 Standards.

Bovine pericardium is used for the examples below. The harvested bovine pericardium may be treated by removing large pieces of fat, muscle and connective tissue, washing to remove blood stains, and then putting into normal saline at 4° C. to be kept under a low temperature in case for experimental use. The pericardium may be carefully chosen and washed, and then randomly divided into two groups to use as the experimental example and the control example.

### Control Example

The chosen bovine pericardium may be immersed into 0.625% glutaraldehyde (GA) solution to undergo the crosslinking reaction.

### Experimental Example

The chosen bovine pericardium may be immersed into a mixed solution of 0.1 mol/L EDC/NHS to undergo the crosslinking reaction.

The crosslinking temperature, crosslinking pressure, and crosslinking time may be identical in the control example and the experimental example.

Performance tests may be carried out on the crosslinked bovine pericardium after the crosslinking reaction is over.

First, testing and comparison of mechanical properties.

One piece of bovine pericardium may be harvested from each of the control example solution and the experimental example solution, and the testing may be carried out according to the testing stands in the GB/T 528-2009 Rubber, vulcanized rubber or thermoplastic, determination of tensile stress-strain properties. The testing samples may be trimmed into dumb bell shape with a laser cutting machine. For each crosslinking pericardium, 20 samples may be obtained for subsequent testing.

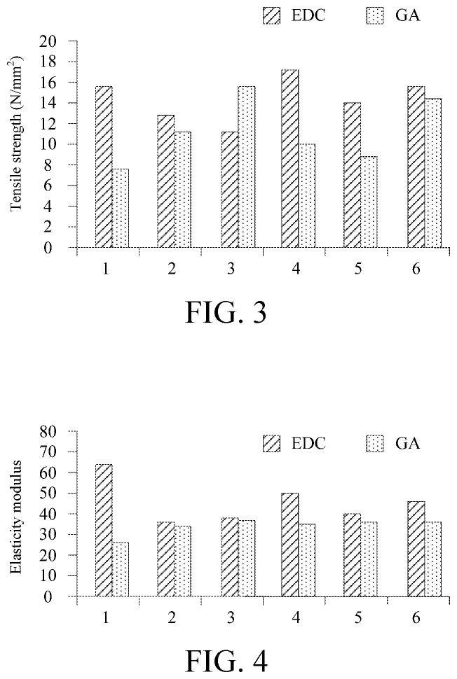

The testing may be finished on a servo material mechanics testing machine under room temperature. The testing length may be set at 10 mm, and the testing sample may be drawn to stretch along the longitudinal direction at a constant speed of 200 mm/min. The software in the system may make a real-time automatic gathering and recording of data, which may include the maximum stress, tensile strength, elastic modulus and the like. Data acquired from 6 samples may be compared, analyzed, and shown in FIGS. 2-4. FIG. 2 is a performance parameter comparison chart of the maximum stress of the crosslinked artificial biological tissue of the present disclosure, FIG. 3 is a performance parameter comparison chart of the tensile strength of the crosslinked artificial biological tissue of the present disclosure, and FIG. 4 is a performance parameter comparison chart of the elasticity modulus of the crosslinked artificial biological tissue of the present disclosure.

The test result may show that the mechanical performance of the samples of EDC crosslinking experiment is higher than that of the samples of glutaraldehyde (GA) crosslinking experiment.

Second, crosslinking index testing.

One piece of bovine pericardium may be harvested from each of the control example solution and the experimental example, and then may be rinsed in normal saline with shaking at 60 rpm for three times, 5 minutes each time. The bovine pericardium may be lyophilized according to MPI 13019 standard, and then 0.2 g sample may be trimmed from the bovine pericardium, cut into pieces to be ready for use. Three samples may be taken from each crosslinked pericardium. Meanwhile, fresh bovine pericardium may be used as blank control, 0.2 g purified water may be used as a negative control, and 0.2 g bovine serum albumin is used as a positive control.

The testing may be carried out according to the experimental protocols for standard crosslinking indexes in EP130015 Measuring experiment protocol of cardiac vesicle crosslinking degree (X1 version), and the testing results are as shown in Table 1.

TABLE 1
The crosslinking index results of the control example
and experimental example of the present disclosure.

| Group | Crosslinking degree |
|---|---|
| Control example | 58.10% |
| Experimental example | 57.60% |

The experimental results may indicate that after the same crosslinking time, the crosslinking degree of glutaraldehyde crosslinking may be slightly higher than that of EDC crosslinking, but the crosslinking degrees do not show static difference (P<0.05).

Third, duration testing.

The bovine pericardium crosslinked with the solution in the experimental example may be obtained and rinsed in normal saline with shaking at 60 rpm for three times, 5 minutes each time. Four valves may be stitched according to the requirements of clinic experimental samples. Tests may be made according to the standard YY/T 1449.3-2016 Cardiovascular implant—Artificial heart valve Part III, Transcatheter implantable prosthetic heart valve. The appearance and fluid dynamic tests may be made after every 25 million stress tests, and at least 50 million tests are made.

The test results are shown in the following.

(1) Appearance: after 200 million duration tests, the valve leaflet may all show different degrees of abrasion, but no abnormal events like valve leaflet tearing, layered swelling, imperfect match of valve leaflets and valve leaflet excessive deformation may have been observed.
(2) The opening and closing of valve leaflets during the pulsating flow test: two EDC crosslinked valves may have been working as expected during the duration test.
(3) Function tests: The effective open area of the two valves in the duration tests may both fit the requirements of YY/T 1449.3-2016 and ISO5840-3:2013. The overall regurgitation ratio and pressure difference may across the valve show a decreasing tendency.

The test results may indicate that the valves that have experienced the duration test show fine functions, and may indicate that the EDC crosslinked bovine pericardium show qualified duration. EDC crosslinking may be thus proven to be applicable.

Fourth, reductive matter test.

One piece of bovine pericardium may be harvested from each of the control example solution and the experimental example, and then may be rinsed in normal saline with shaking at 60 rpm for three times, 5 minutes each time. Physical and chemical tests may be made after rinsing.

Tests may be carried out according to the testing methods in section 5.2.2 of GB/T 14233.1-2008, and the concentration of the potassium permanganate may be [(KMnO₄)=0.002 mol/L]. The testing results are shown in Table 2.

TABLE 2
The reductive matter testing results of the control example
and experimental example of the present disclosure.

| | The initial volume | The final volume | difference |
|---|---|---|---|
| | of potassium | of potassium | of volume |
| | permanganate mL | permanganate mL | (ΔV)ml |
| Blank (sterile | 1.4 | 4.5 | 3.1 |
| water for injection) | | | |
| Glutaraldehyde | 10.4 | 18.9 | 8.5 |
| crosslinked bovine | | | |
| pericardium | | | |
| EDC crosslinked | 4.6 | 10.3 | 5.7 |
| bovine | | | |
| pericardium | | | |

The test results show that the reductive matter contained in the EDC crosslinked bovine pericardium may be far lower than that contained in the glutaraldehyde crosslinked bovine pericardium.

Fifth, Cytotoxicity test.

One piece of bovine pericardium may be harvested from each of the control example solution and the experimental example, and then may be lyophilized, EO sterilization and coerced to resolve. Four grams may be taken for cytotoxicity tests, and three parallel tests are carried out.

Testing solution may be prepared by digesting at 0.2 g/mL, (37±1) ° C., and (24±2) h. The digesting medium may be a cell culture fluid containing serum. The testing solution may be obtained according to the method in section 8.2 of GB/T16886.5-2003. The testing results are shown in Table 3, and the cytotoxic criteria are shown in Table 4.

TABLE 3
The cell viability testing results of the control example
and experimental example of the present disclosure.

| Group | X ± SD | Cell Viability % |
|---|---|---|
| Blank Control | 0.480 ± 0.043 | 100.0% |
| Negative Control | 0.448 ± 0.018 | 93.3% |
| Positive Control | 0.028 ± 0.005 | 5.9% |
| 100% Sample Digest Solution | 0.361 ± 0.006 | 75.2% |
| 75% Sample Digest Solution | 0.403 ± 0.016 | 83.9% |
| 50% Sample Digest Solution | 0.403 ± 0.016 | 83.9% |
| 25% Sample Digest Solution | 0.411 ± 0.009 | 85.6% |

TABLE 4
Cytotoxicity criteria

| | Cell Viability | Cytotoxicity Level |
|---|---|---|
| 1 | 100% | Level 0 |
| 2 | 75~99% | Level 1 |
| 3 | 50~74% | Level 2 |
| 4 | 25~49% | Level 3 |
| 5 | ≤24% | Level 4 or Level 5 |

The test result shows that the EDC crosslinked sample, which may be produced under laboratory conditions, lyophilized, EO sterilized, and coerced to resolve, shows a cell viability of 75.2%, and level 1 cytotoxicity, and it may satisfy the standard for acceptance.

Sixth, anti-calcification test.

The bovine pericardium with a thickness of 0.2-0.3 cm in the control example solution and experimental example solution may be obtained respectively, and may be cut into 10 mm*10 mm pieces with a laser cutter machine. Thirty pieces may be trimmed from each sample. The samples treated with glutaraldehyde may be immersed into a sterilizing solution to be ready for use. EDC treated sample may be lyophilized and packed with dialysis bags, and may be sterilized with ethylene oxide to be ready for use. The groups and the number of samples are shown in Table 5.

TABLE 5
Treating conditions of the calcification samples of the control
examples and experimental examples of the present disclosure.

| Treatment of the Experimental Samples | | Number | Duration |
|---|---|---|---|
| Control Group | Anti-calcification Group | of Rats | of Implant |
| Glutaraldehyde cross- | EDC crosslinked; | 10 | 8 weeks |
| linked; lyophilized for 16 | lyophilized for 16 | | |
| hours + EO sterilized | hours + EO sterilized | | |

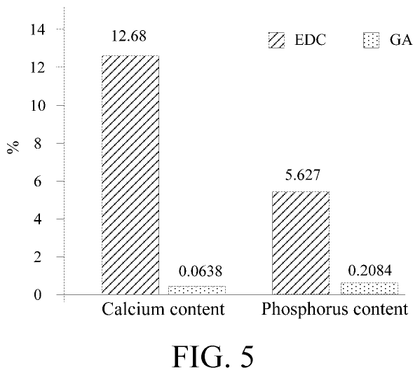

Rat hypodermic implantation may be carried out according to the protocols in TP13010, and the testing results are shown in Table 6 and FIG. 5. FIG. 5 is a performance parameter comparison chart of the anti-calcification capability of the crosslinked artificial biological tissue of the present disclosure.

TABLE 6
The results of the anti-calcification test of the control examples
and experimental examples of the present disclosure.

| | Calcium Content (%) | | Phosphorus Content | |
|---|---|---|---|---|
| | Control | Anti-cal- | Control | Anti-cal- |
| | Sample | cification | Sample | cification |
| Number | (Glutar- | Sample | (Glutar- | Sample |
| of | aldehyde | (EDC | aldehyde | (EDC |
| Animals | crosslinked) | crosslinked | crosslinked) | crosslinked |
| 10 | 12.68 ± | 0.0638 ± | 5.62702 ± | 0.2084 ± |
| | 2.777 | 0.018 | 1.116 | 0.058 |

The test results may indicate that, the average calcium and phosphorus contents in EDC crosslinked bovine pericardium samples may be significantly lower than those in the glutaraldehyde crosslinked pericardium samples. The calcium content in EDC crosslinked pericardium samples may be lower than 0.84%, and the phosphorus content in EDC crosslinked pericardium samples may be lower than 1.94%.

It may be concluded from the above that EDC may well replace glutaraldehyde to crosslink and immobilize bovine pericardium, and may be applied in artificial heart valves. The present disclosure may only use EDC solution for crosslinking, and no aldehyde crosslinking agent may be introduced. Therefore, the calcification risks brought by aldehydes may have been avoided from the very beginning.

In conclusion of the above technical solutions, the present disclosure may provide a method for crosslinking an artificial biological tissue. The crosslinking agents used in the crosslinking method may be crosslinking agents with imide structures, and do not produce calcification sites as aldehyde crosslinking agents do, thereby the anti-calcification capacity of the crosslinked artificial biological tissue may be enhanced. Meanwhile, the crosslinked artificial biological tissue may possess good physical and chemical properties, as well as good biocompatibility.

It is understood that the descriptions above are only embodiments of the present disclosure. It is not intended to limit the scope of the present disclosure. Any equivalent transformation in structure and/or in scheme referring to the instruction and the accompanying drawings of the present disclosure, and direct or indirect application in other related technical field, are included within the scope of the present disclosure.

What is claimed is:

1. A method for crosslinking an artificial biological tissue, comprising:
providing an artificial biological tissue and a crosslinking agent solution, wherein the crosslinking agent solution comprises an imide structure;
immersing the artificial biological tissue in the crosslinking agent solution to produce a crosslinking reaction.

2. The method according to claim 1, wherein the crosslinking agent solution is 1-ethyl-3-(3-dimethylaminopropyl) carbodiimide and N-hydroxy succinimide.

3. The method according to claim 2, wherein a mass fraction of the 1-ethyl-3-(3-dimethylaminopropyl) carbodiimide in the crosslinking agent solution is 0.5 g/L˜30 g/L.

4. The method according to claim 2, wherein a mass fraction of the N-hydroxy succinimide in the crosslinking agent solution is 0.1 g/L˜10 g/L.

5. The method according to claim 1, wherein a crosslinking temperature of the crosslinking reaction is between 25° C. and 45° C.

6. The method according to claim 1, wherein a crosslinking pressure of the crosslinking reaction ranges from 0 mm Hg to 40 mm Hg.

7. The method according to claim 1, wherein the crosslinking reaction is performed on a crosslinking machine with a motion amplitude between 10 rpm and 100 rpm.

8. The method according to claim 1, wherein a crosslinking time of the crosslinking reaction is between 0.5 day and 60 days.

9. The method according to claim 1, wherein the artificial biological tissue is a mammal tissue.

10. The method according to claim 9, wherein the mammal tissue is any one of an animal pericardium, an aortic valve, a mitral valve, a tricuspid valve, a pulmonary valve, a ligament, a skin, a peritoneum, a pleura, a heel tendon or a venous valve, or a mixture of at least two selected from the above.
